(19) United States
(12) Patent Application Publication
(10) Pub. No.: US 2014/0163097 A1
(43) Pub. Date: Jun. 12, 2014

(54) ANIMAL SUPPLEMENTS AND FOOD COMPOSITIONS CONTAINING SOLUBLE MONENSIN COMPOSITION, AND METHODS AND PROCESSES THEREFOR

(71) Applicants: Jon Thomas Gawlak, Athens, AL (US); Steven Duff Lubetkin, Zionsville, IN (US)

(72) Inventors: Jon Thomas Gawlak, Athens, AL (US); Steven Duff Lubetkin, Zionsville, IN (US)

(73) Assignee: ELI LILLY AND COMPANY, Indianapolis, IN (US)

(21) Appl. No.: 14/232,697

(22) PCT Filed: Jul. 26, 2012
(86) PCT No.: PCT/US2012/048215

§ 371 Date: Jan. 14, 2014

Related U.S. Application Data
(60) Provisional application No. 61/515,451, filed on Aug. 5, 2011.
(60) Provisional application No. 61/515,457, filed on Aug. 5, 2011.

(51) Int. Cl. A61K 31/352; A23K 1/17

(52) U.S. Cl. CPC A61K 31/352; A23K 1/17
USPC 514/460

(57) ABSTRACT

The present disclosure provides animal feed supplements comprising a therapeutically effective amount of monensin, a solvent, and a mineral mix, wherein the mineral mix is substantially free of monocalcium phosphate. In addition, the present disclosure provides processes of preparing animal feed supplements and animal food compositions comprising such animal feed supplements. The present disclosure provides animal feed supplements comprising a therapeutically effective amount of monensin, a solvent, and an alkaline component. In addition, the present disclosure provides animal food compositions containing such animal feed supplements, and methods for their use.

This application claims the benefit under 35 USC §119(e) of U.S. Provisional Application Ser. No. 61/515,451, filed on Aug. 5, 2011, and U.S. Provisional Application Ser. No. 61/515,457, filed on Aug. 5, 2011. The entire disclosures of both U.S. Provisional Application Ser. No. 61/515,451 and U.S. Provisional Application Ser. No. 61/515,457 are incorporated herein by reference.

Monensin is an ionophore antibiotic isolated from the bacteria Streptomyces cinnamonensis. In veterinary medicine, monensin can be advantageously administered to animals for a variety of purposes. For example, administration of a therapeutically effective dose of monensin can be utilized for the treatment or prevention of ketosis and/or bloat, for the enhancement of milk production efficiency, for the enhancement of milk protein content in milk, for the enhancement of mineral uptake, for the enhancement of weight gain, for the enhancement of feed conversion efficiency, and the provision of desirable reproduction advantages.

Monensin is typically administered via a solid formulation that is ingested by an animal. For instance, a solid formulation of monensin can be a combined with a mineral mix (for example, a solid mineral mix) and/or an animal feed, followed by consumption of the combination by an animal. For bovines, the daily intake of monensin ingestion is targeted at about 50 milligrams (mg) to about 500 mg per head per day.

However, when a solid formulation of monensin is combined with a mineral mix and/or an animal feed, the amount of the combination ingested by an animal, particularly a bovine, is typically decreased compared to combinations that do not contain monensin. Without being bound by any theory, it is hypothesized that the solid formulation of monensin may have a taste which the animal prefers less than the monensin-free formulation. As a result, the animal may reduce its intake and ingestion of the monensin-containing combination. As hypothesized, the taste of the solid monensin formulation could be problematic to veterinary practice. For instance, the taste could decrease the intake of monensin itself and thus inhibit its therapeutic effectiveness. In addition, the taste could lower the consumption of the combined mineral mixes and feed, thus potentially contributing to myriad other problems in animals due to the decreased intake of nourishment.

Therefore, there exists a need for compositions and methods utilizing monensin, and processes directed thereto, that overcomes the limitations of current solid compositions and formulations in order to benefit the intake of monensin, minerals, feeds, and foodstuffs by animals.

Moreover, alternative formulations of monensin, such as some soluble formulations, can potentially cause difficulties for the assayed detection of monensin following combination with a mineral mix. Mineral mixes typically contain a variety of minerals, often including monocalcium phosphate. For example, when monocalcium phosphate and monensin (e.g., monensin sodium) are both present in a liquid environment, a complex can form between the two substances, thus resulting in a reduction of assayed detection of monensin in the mixture. It has been hypothesized that monocalcium phosphate binds to and degrades monensin, resulting in low analytical recovery and low extractability of the monensin. Therefore, there exists a need for process to prepare an animal feed supplement containing monensin that overcomes the limitations reduced detection of monensin following combination of monensin, a solvent, and a mineral mix.

Accordingly, the present disclosure provides utilization of a formulation of monensin that exhibits desirable properties and provides related advantages for its intake in animals, as well as preventing the reduction in detection of monensin previously observed in a liquid environment.

Furthermore, the present disclosure describes a process for preparing an animal feed supplement containing a therapeutically effective amount of monensin in a soluble composition that exhibits desirable properties and provides related advantages for its intake in animals, as well as preventing the reduction in detection of monensin previously obtained by alternative processes.

The present disclosure demonstrates that the reduced intake of monensin can be overcome in animals by utilizing a formulation comprising monensin, a solvent, and an alkaline component. In addition, present disclosure demonstrates that the reduced intake of monensin can be overcome in animals by preparing an animal feed supplement containing a therapeutically effective amount of monensin as a soluble composition. By combining the formulation or soluble composition of monensin with a mineral mix and/or an animal feed, the present invention overcomes the potential limitations of monensin's taste when prepared or used as a solid formulation. Furthermore, the present disclosure demonstrates that the soluble formulation of monensin can be prepared and utilized without the reduction in detection of monensin.

The present disclosure provides an animal feed supplement comprising a therapeutically effective amount of monensin, a solvent, and a mineral mix, wherein the mineral mix is substantially free of monocalcium phosphate. In addition, the present disclosure provides processes of preparing such animal feed supplements comprising the step of combining a mineral mix and a soluble composition comprising a therapeutically effective amount of monensin and a solvent, wherein the mineral mix is substantially free of monocalcium phosphate.

The present disclosure also provides animal feed supplements comprising a therapeutically effective amount of monensin, a solvent, and an alkaline component, wherein the supplement is a mixture. Furthermore, the disclosure provides food compositions comprising a therapeutically effective amount of monensin, a solvent, an alkaline component, and an animal feed, methods of administering the food compositions, and processes for making the food compositions.

The monensin-containing animal feed supplements and food compositions, and the processes used to prepare them, according to the present disclosure provide several advantages compared to the supplements, compositions, and processes using conventional techniques. First, the monensin-containing supplements and compositions of the present disclosure allow for increased intake of monensin by animals compared to products using a solid monensin formulation. Second, the ingestion of mineral mixes and animal feeds by animals are may also be increased compared to those using a solid monensin formulation. These advantages could be the result of the hypothesized improved taste of the monensin-containing animal feed supplements and food compositions according to the present disclosure.

Third, the monensin-containing supplements and compositions of the present disclosure are easier to mix homogenously, thus resulting in better spreading and minimized segregation of the product compared to those using a solid monensin formulation. Fourth, the inclusion of an alkaline component in the monensin-containing supplements and compositions of the present disclosure maintains the ability to detect monensin in the formulation. Fifth, the monensin-containing supplements of the present disclosure may be prepared as solutions without the requirement of complex additional ingredients and can be easily combined with a mineral mix and/or an animal feed. Sixth, the monensin-containing compositions of the present disclosure can be prepared easily and inexpensively compared to preparations using the solid monensin formulation, and could potentially be prepared on-site at the place of consumption. Finally, the process of preparing the animal feed supplements and food compositions maintains the ability to detect monensin in the supplements and compositions compared to alternative procedures generally used in the art.

In some illustrative embodiments described herein, an animal feed supplement is provided. The animal feed supplement comprises a) a mineral mix and b) a soluble composition comprising a therapeutically effective amount of monensin and a solvent, wherein the mineral mix is substantially free of monocalcium phosphate. In some embodiments, a process for preparing an animal feed supplement is provided. The process comprises the step of combining a) a mineral mix and b) a soluble composition comprising a therapeutically effective amount of monensin and a solvent, wherein the mineral mix is substantially free of monocalcium phosphate.

In other illustrative embodiments described herein, an animal food composition is provided. The animal food composition comprises a) an animal feed and b) a soluble composition comprising a therapeutically effective amount of monensin and a solvent, wherein the animal feed is substantially free of monocalcium phosphate. In some embodiments, a process for preparing an animal food composition is provided. The process comprises the step of combining a) an animal feed and b) a soluble composition comprising a therapeutically effective amount of monensin and a solvent.

In some illustrative embodiments described herein, an animal feed supplement is provided. The animal feed supplement comprises a therapeutically effective amount of monensin, a solvent, and an alkaline component, wherein the supplement is a mixture. In other illustrative embodiments, a food composition is provided. The food composition comprises a therapeutically effective amount of monensin, a solvent, an alkaline component, and an animal feed. In some embodiments, a process for preparing a food composition is provided. The process comprises combining a) a mixture comprising monensin and a solvent, b) an alkaline component, and c) an animal feed.

In some illustrative embodiments described herein, a method of improving feed efficiency of an animal is provided. In other illustrative embodiments, a method of increasing milk production efficiency of an animal is provided. In yet other illustrative embodiments, a method of increasing rate of weight gain of an animal is provided. In some illustrative embodiments, a method of preventing or treating of coccidiosis in an animal is provided. The described methods comprise administering to an animal a food composition comprising a therapeutically effective amount of monensin, a solvent, an alkaline component, and an animal feed.

Various embodiments of the present application utilize a soluble composition containing monensin and a solvent. As used herein, the term “solution” refers to a substantially homogenous one-phase system of two or more substances, for example a solute and a solvent. As used herein, the term “solute” refers to the dissolving phase of a solution. In some embodiments, the solute is monensin.

Various embodiments of the present application utilize an animal feed supplement containing monensin and a solvent, wherein the supplement is a mixture. As used herein, the term “mixture” refers to solutions, suspensions, emulsions, and the like that are well known in the art.

As used herein, the term “solvent” refers to the dispersing medium of a mixture. In some embodiments, the solvent is a liquid at standard temperature and pressure, and may be capable of solubilizing an appreciable amount of a specified solid. Solids vary from 0-100% in their degree of solubility. See, e.g., “Solubility Parameters of Organic Compounds,” CRC Handbook of Chemistry and Physics, 62d ed., C-699, CRC Press; N. Irving Sax and Richard J. Lewis, Sr., Hawley's Condensed Chemical Dictionary, 11th ed., 1079 (1987). The term “solvent” also includes combinations of two or more solvents.

As used herein, the term “monensin” refers to monensin base, pharmaceutically acceptable salts of monensin, or other salts of monensin. The term “pharmaceutically acceptable salt” refers to an addition salt that exists in conjunction with the acidic or basic portion of monensin. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. In some embodiments, “monensin” is monensin sodium.

Pharmaceutically acceptable salts of an acid addition nature are formed when monensin and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when monensin and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present invention. They may serve as intermediates in the purification of compounds or in the preparation of other pharmaceutically-acceptable salts, or are useful for identification, characterization or purification.

Solvents according to the present disclosure are pharmaceutically acceptable for ingestion by an animal. A number of pharmaceutically acceptable solvents are known in the art. In some embodiments, the solvent is selected from the group consisting of benzyl alcohol, oleic acid, propylene glycol, vitamin E, ethanol, glyceryl mono- and di-caprylate, and mixtures thereof. In other embodiments, the solvent is benzyl alcohol. In other embodiments, the solvent is oleic acid. In other embodiments, the solvent is a derivatized propylene glycol (e.g., propylene glycol monocaprylate (Capryol 90™, Gattefossé Canada Inc). In other embodiments, the solvent is vitamin E. In other embodiments, the solvent is ethanol. In other embodiments, the solvent is glyceryl mono- and di-caprylate (i.e., Capmul MCM™, ABITEC Corporation).

The animal feed supplements described herein may contain an alkaline component. The presence of an alkaline component in the animal feed supplement is believed to maintain the ability perform assayed detection of monensin in the formulation. Without being bound by any theory, monocalcium phosphate in a mineral mix is hypothesized to potentially complex with monensin, thus leading to the decreased assayed detection of monensin in the formulation.

As used herein, the term “alkaline component” refers to a basic salt of an alkali metal or alkaline earth metal. A number of alkaline components are known in the art. In some embodiments, the alkaline component is selected from the group consisting of calcium hydroxide, sodium hydroxide, potassium hydroxide, and mixtures thereof. In one embodiment, the alkaline component is calcium hydroxide. In one embodiment, the alkaline component is sodium hydroxide. In one embodiment, the alkaline component is potassium hydroxide.

The amount of monensin in the supplement is adequate to achieve a therapeutic effect. As used herein, the term “therapeutically effective amount” refers to an amount which gives the desired benefit to an animal and includes both treatment and prophylactic administration. The amount will vary from one individual to another and will depend upon a number of factors, including the overall physical condition of the animal and the underlying cause of the condition to be treated. The amount of monensin used for therapy gives an acceptable rate of change and maintains desired response at a beneficial level. A therapeutically effective amount of the present supplements may be readily ascertained by one of ordinary skill in the art using publicly available materials and procedures.

In some embodiments of the present disclosure, the amount of monensin in the supplement can vary. For example, in some embodiments, the amount of monensin can be present in the supplement in an amount of between about 5% to about 40% (weight of monensin/(weight of monensin plus solvent)). In some embodiments, the amount of monensin can be present in the supplement in an amount of between about 5% to about 35% (weight of monensin/(weight of monensin plus solvent)). In some embodiments, the amount of monensin can be present in the supplement in an amount of between about 5% to about 30% (weight of monensin/(weight of monensin plus solvent)). In some embodiments, the amount of monensin can be present in the supplement in an amount of between about 5% to about 25% (weight of monensin/(weight of monensin plus solvent)). In some embodiments, the amount of monensin can be present in the supplement in an amount of between about 5% to about 20% (weight of monensin/(weight of monensin plus solvent)). In some embodiments, the amount of monensin can be present in the supplement in an amount of between about 10% to about 20% (weight of monensin/(weight of monensin plus solvent)). In some embodiments, the amount of monensin is present in the supplement in an amount of about 25% (weight of monensin/(weight of monensin plus solvent)). In some embodiments, the amount of monensin is present in the supplement in an amount of about 20% (weight of monensin/(weight of monensin plus solvent)). In some embodiments, the amount of monensin is present in the supplement in an amount of about 15% (weight of monensin/(weight of monensin plus solvent)). In some embodiments, the amount of monensin is present in the supplement in an amount of about 10% (weight of monensin/(weight of monensin plus solvent)). In some embodiments, the amount of monensin is present in the supplement in an amount of about 5% (weight of monensin/(weight of monensin plus solvent)).

The amount of monensin in the supplement is a sufficient amount to provide animals, such as bovines, with between about 50 mg to about 500 mg per head per day. In some embodiments, the amount of monensin in the supplement provides animals with between about 100 mg of monensin to about 400 mg of monensin per day. In some embodiments, the amount of monensin in the supplement provides animals with about 100 mg of monensin per day. In some embodiments, the amount of monensin in the supplement provides animals with about 200 mg of monensin per day. In some embodiments, the amount of monensin in the supplement provides animals with about 250 mg of monensin per day. In some embodiments, the amount of monensin in the supplement provides animals with about 300 mg of monensin per day.

In some embodiments of the present disclosure, the amount of solvent in the supplement can vary. For example, in some embodiments, the amount of solvent can be present in the supplement in an amount of between about 60% to about 95% (weight of solvent/(weight of solvent plus monensin)). For example, in some embodiments, the amount of solvent can be present in the supplement in an amount of between about 70% to about 95% (weight of solvent/(weight of solvent plus monensin)). For example, in some embodiments, the amount of solvent can be present in the supplement in an amount of between about 75% to about 95% (weight of solvent/(weight of solvent plus monensin)). For example, in some embodiments, the amount of solvent can be present in the supplement in an amount of between about 80% to about 95% (weight of solvent/(weight of solvent plus monensin)). In some embodiments, the amount of solvent can be present in the supplement in an amount of between about 80% to about 90% (weight of solvent/(weight of solvent plus monensin)). In some embodiments, the amount of solvent is present in the supplement in an amount of 75% (weight of solvent/(weight of solvent plus monensin)). In some embodiments, the amount of solvent is present in the supplement in an amount of 80% (weight of solvent/(weight of solvent plus monensin)). In some embodiments, the amount of solvent is present in the supplement in an amount of 85% (weight of solvent/(weight of solvent plus monensin)). In some embodiments, the amount of solvent is present in the supplement in an amount of 90% (weight of solvent/(weight of solvent plus monensin)). In some embodiments, the amount of solvent is present in the supplement in an amount of 95% (weight of solvent/(weight of solvent plus monensin)).

In other embodiments of the present disclosure, the amount of solvent in the supplement can vary. For example, in some embodiments, the amount of solvent can be present in the supplement in an amount of between about 50% to about 94.9% (weight of solvent/(weight of solvent plus monensin plus alkaline component)). In some embodiments, the amount of solvent can be present in the supplement in an amount of between about 55% to about 94.9% (weight of solvent/(weight of solvent plus monensin plus alkaline component)). In some embodiments, the amount of solvent can be present in the supplement in an amount of between about 60% to about 94.9% (weight of solvent/(weight of solvent plus monensin plus alkaline component)). In some embodiments, the amount of solvent can be present in the supplement in an amount of between about 65% to about 94.9% (weight of solvent/(weight of solvent plus monensin plus alkaline component)). In some embodiments, the amount of solvent can be present in the supplement in an amount of between about 70% to about 94.9% (weight of solvent/(weight of solvent plus monensin plus alkaline component)). In some embodiments, the amount of solvent can be present in the supplement in an amount of between about 70% to about 89.9% (weight of solvent/(weight of solvent plus monensin plus alkaline component)). In some embodiments, the amount of solvent is present in the supplement in an amount of 73% (weight of solvent/(weight of solvent plus monensin plus alkaline component)). In some embodiments, the amount of solvent is present in the supplement in an amount of 78% (weight of solvent/(weight of solvent plus monensin plus alkaline component)). In some embodiments, the amount of solvent is present in the supplement in an amount of 83% (weight of solvent/(weight of solvent plus monensin plus alkaline component)). In some embodiments, the amount of solvent is present in the supplement in an amount of 88% (weight of solvent/(weight of solvent plus monensin plus alkaline component)). In some embodiments, the amount of solvent is present in the supplement in an amount of 93% (weight of solvent/(weight of solvent plus monensin plus alkaline component)).

In some embodiments of the present disclosure, the amount of the alkaline component in the supplement can vary. For example, in some embodiments, the amount of the alkaline component can be present in the supplement in an amount of between about 0.1% to about 10% (weight of alkaline component/(weight of alkaline component plus monensin plus solvent)). In some embodiments, the amount of the alkaline component can be present in the supplement in an amount of between about 1% to about 8% (weight of alkaline component/(weight of alkaline component plus monensin plus solvent)). In some embodiments, the amount of the alkaline component can be present in the supplement in an amount of between about 2% to about 6% (weight of alkaline component/(weight of alkaline component plus monensin plus solvent)). In some embodiments, the amount of the alkaline component can be present in the supplement in an amount of between about 3% to about 5% (weight of alkaline component/(weight of alkaline component plus monensin plus solvent)). In some embodiments, the amount of the alkaline component can be present in the supplement in an amount of between about 0.1% to about 1% (weight of alkaline component/(weight of alkaline component plus monensin plus solvent)). In some embodiments, the amount of the alkaline component is present in the supplement in an amount of about 0.1% (weight of alkaline component/(weight of alkaline component plus monensin plus solvent)). In some embodiments, the amount of the alkaline component is present in the supplement in an amount of about 0.5% (weight of alkaline component/(weight of alkaline component plus monensin plus solvent)). In some embodiments, the amount of the alkaline component is present in the supplement in an amount of about 1% (weight of alkaline component/(weight of alkaline component plus monensin plus solvent)). In some embodiments, the amount of the alkaline component is present in the supplement in an amount of about 2% (weight of alkaline component/(weight of alkaline component plus monensin plus solvent)). In some embodiments, the amount of the alkaline component is present in the supplement in an amount of about 5% (weight of alkaline component/(weight of alkaline component plus monensin plus solvent)). In some embodiments, the amount of the alkaline component is present in the supplement in an amount of about 10% (weight of alkaline component/(weight of alkaline component plus monensin plus solvent)).

In some embodiments described in the present disclosure, the supplements are associated with an improvement in consumption of the supplement. As used herein, the term “improvement in consumption” can refer to an increase in the amount of monensin consumed by an animal fed the supplement of the present disclosure compared to the amount of monensin consumed by an animal fed a solid formulation of monensin. In some embodiments, the supplement is consumed by an animal at a rate of about 50 mg of monensin to about 500 mg of monensin per day. In some embodiments, the supplement is consumed by an animal at a rate of about 100 mg of monensin to about 400 mg of monensin per day. In some embodiments, the supplement is consumed by an animal at a rate of about 100 mg of monensin per day. In some embodiments, the supplement is consumed by an animal at a rate of about 200 mg of monensin per day. In some embodiments, the supplement is consumed by an animal at a rate of about 250 mg of monensin per day. In some embodiments, the supplement is consumed by an animal at a rate of about 300 mg of monensin per day.

In another embodiment of the present disclosure, a food composition comprising a therapeutically effective amount of monensin, a solvent, an alkaline component, and an animal feed is described. In other embodiments described in the present disclosure, the animal feed supplement is subsequently combined with an animal feed. As used herein, the term “animal feed” refers to a composition that can be ingested by an animal, such as an animal feedstuff. In some embodiments, the animal feed is an animal feed pellet. The solvents, concentrations of monensin (i.e., weight of monensin/(weight of monensin plus solvent plus alkaline component)), concentrations of solvent (i.e., weight of solvent/(weight of solvent plus monensin plus alkaline component)), concentrations of alkaline component (i.e., weight of alkaline component/(weight of alkaline component plus monensin plus solvent)), amounts of monensin, amounts of consumption, and improvement in consumption provided in the preceding paragraphs are also applicable to the food compositions described herein.

In some embodiments described in the present disclosure, the supplement is combined with a mineral mix. As used herein, the term “mineral mix” refers to a combination of at least one known mineral with at least one other known mineral. In one embodiment, a mineral mix comprises sodium chloride, calcium carbonate, a carrier, and a number of minor nutrient species.

In some embodiments, the mineral mix is substantially free of monocalcium phosphate. As used herein, the term “substantially free of monocalcium phosphate” means that the mineral mix comprises trace quantities of monocalcium phosphate. In some embodiments, the mineral mix comprises from about 0.1% to about 5% monocalcium phosphate. In some embodiments, the mineral mix comprises about 5% or less monocalcium phosphate. In some embodiments, the mineral mix comprises about 2% or less monocalcium phosphate. In some embodiments, the mineral mix comprises about 1% or less monocalcium phosphate. In some embodiments, the mineral mix comprises about 0.5% or less monocalcium phosphate. In some embodiments, the mineral mix comprises about 0.1% or less monocalcium phosphate.

In some embodiments, the food composition contains a solvent wherein the solvent is present in a residual amount. As used herein, the term “residual” refers to the remaining portion of a solvent present in the food composition after an initial portion of the solvent has evaporated. For example, if the solvent is a relatively non-volatile solvent (e.g., benzyl alcohol), it will tend evaporate slowly after combination and may be present at a greater amount in the food composition. However, if the solvent is a relatively volatile solvent (e.g., ethanol), it will tend to evaporate quickly after combination and a residual amount will be present in the food composition. In some embodiments, a residual amount can be an amount of solvent that is about 0.1% to about 10% of the original amount of solvent initially present in the food composition. In other embodiments, a residual amount can be an amount of solvent that is about 0.1% to about 1% of the original amount of solvent initially present in the food composition. In other embodiments, a residual amount can be an amount of solvent that is about 1% to about 8% of the original amount of solvent initially present in the food composition. In other embodiments, a residual amount can be an amount of solvent that is about 4% to about 6% of the original amount of solvent initially present in the food composition. In other embodiments, a residual amount can be an amount of solvent that is about 5% of the original amount of solvent initially present in the food composition. In other embodiments, a residual amount can be an amount of solvent that is about 2.5% of the original amount of solvent initially present in the food composition. In other embodiments, a residual amount can be an amount of solvent that is about 1% of the original amount of solvent initially present in the food composition. In other embodiments, a residual amount can be an amount of solvent that is about 0.1% of the original amount of solvent initially present in the food composition. In other embodiments, a residual amount can be an amount of solvent that is less than 0.1% of the original amount of solvent initially present in the food composition.

In some embodiments, the soluble composition of monensin is allowed to dry following combination with the mineral mix. In some embodiments, monocalcium phosphate can be subsequently combined with the soluble composition and the mineral mix. In other embodiments, the monocalcium phosphate that is combined with the soluble composition and the mineral mix is dry.

In other embodiments described in the present disclosure, various methods comprising administering to an animal in need thereof a food composition comprising a therapeutically effective amount of monensin, a solvent, an alkaline component, and an animal feed are described. As used herein, the term “administered” is used in its broadest sense and refers to any method of delivering a substance to an animal. In some embodiments, the food composition is administered to an animal via consumption by the animal. As used herein, the term “consumption” refers to the intake or ingestion of a substance by an animal, for example by eating the substance. The solvents, concentrations of monensin (i.e., weight of monensin/(weight of monensin plus solvent plus alkaline component)), concentrations of solvent (i.e., weight of solvent/(weight of solvent plus monensin plus alkaline component)), concentrations of alkaline component (i.e., weight of alkaline component/(weight of alkaline component plus monensin plus solvent)), amounts of monensin, amounts of consumption, and improvement in consumption provided in the preceding paragraphs are also applicable to the methods described herein.

In some embodiments, a method of improving feed efficiency is described. As used herein, the term “improving feed efficiency” refers to an improvement in the ratio of unit of feed/forage consumed to unit of animal weight gain (i.e., unit of feed/forage consumed: unit of animal weight gain) over a specific time period.

In some embodiments, a method of increasing milk production efficiency is described. As used herein, the term “increasing milk production efficiency” refers to an increase in animal production of marketable solids per unit of feed intake.

In some embodiments, a method of increasing rate of weight gain is described. As used herein, the term “increasing rate of weight gain” refers to an increase in the ratio of unit of animal weight gain to unit of time (i.e., unit of animal weight gain:unit of time) over a specific time period.

In some embodiments, a method of preventing or treating of coccidiosis is described. “Preventing” refers to reducing the likelihood that the patient will incur or develop any of the pathological conditions described herein and includes prophylactic administration. The term “preventing” is particularly applicable to a patient that is susceptible to the particular pathological condition. “Treating” refers to mediating a disease or condition and preventing, reversing the clinical effects of the disease, mitigating its further progression, or ameliorating the symptoms associated with the disease or condition.

As used herein, the term “coccidiosis” refers to a parasitic disease of the intestinal tract caused by protozoans. In some embodiments, the coccidiosis is caused by a species from the genus Eimeria. In one embodiment, the coccidiosis is caused by Eimeria bovis. In one embodiment, the coccidiosis is caused by Eimeria zuernii. In one embodiment, the coccidiosis is caused by Eimeria crandallis. In one embodiment, the coccidiosis is caused by Eimeria christenseni. In one embodiment, the coccidiosis is caused by Eimeria ninakohlyakimovae.

In some embodiments, the food composition is administered to a ruminant. In other embodiments, the animal feed supplement is adapted for consumption by a ruminant. As used herein, the term “consumption” refers to the intake or ingestion of a substance by an animal, for example by eating the substance. As used herein, the term “ruminant” refers to an even-toed hoofed animal that has a complex 3-chamber or 4-chamber stomach and which typically re-chews what it has previously swallowed. Some non-exhaustive examples of ruminants include bovines, sheep, goats, oxen, muskox, llamas, alpacas, guanicos, deer, bison, antelopes, camels, and giraffes. In one embodiment, the ruminant is a bovine. In another embodiment, the ruminant is a goat.

In some embodiments, the food composition is administered to an avian. In other embodiments, the animal feed supplement is adapted for consumption by an avian. As used herein, the term “avian” refers to a warm-blooded, egg laying, feathered vertebrate provided with wings, for example birds of any known species or type. In some embodiments, avians include poultry. As used herein, the term “poultry” means any domestic fowl reared for the table, or their eggs or feathers including chickens, (for example, White Leghorn, Brown Leghorn, Barred-Rock, Sussex, New Hampshire, Rhode Island, Ausstralorp, Minorca, Amrox, Calif. Gray, Italian Partidge-colored, etc.), broilers, fryers, cocks and hens, capons, turkeys, ducks, geese, pheasants, quails, ostriches and other poultry commonly bred in commercial quantities.

In other embodiments described in the present disclosure, a process for preparing an animal food composition comprising the step of combining a) an animal feed and b) a soluble composition comprising a therapeutically effective amount of monensin and a solvent, wherein the animal feed is substantially free of monocalcium phosphate is provided. In some embodiments, the soluble composition is allowed to dry following combination with the animal feed. In some embodiments, the animal food composition is subsequently combined with monocalcium phosphate. In various embodiments, the monocalcium phosphate is a solid. The solvents, concentrations of monensin (i.e., weight of monensin/weight of monensin plus solvent), concentrations of solvent (i.e., weight of solvent/weight of solvent plus monensin), amounts of monensin, and adaptation for consumption provided in the preceding paragraphs are also applicable to the processes described herein.

In other embodiments described in the present disclosure, a process for preparing a food composition comprising combining a) a mixture comprising monensin and a solvent, b) an alkaline component, and c) an animal feed is described. In some embodiments, the process further comprises combining a), b), and c) with d) a mineral mix. The solvents, concentrations of monensin (i.e., weight of monensin/(weight of monensin plus solvent plus alkaline component)), concentrations of solvent (i.e., weight of solvent/(weight of solvent plus monensin plus alkaline component)), concentrations of alkaline component (i.e., weight of alkaline component/(weight of alkaline component plus monensin plus solvent)), amounts of monensin, amounts of consumption, and improvement in consumption provided in the preceding paragraphs are also applicable to the processes described herein.

In other embodiments described in the present disclosure, a process for preparing an animal food composition comprising the step of combining a) an animal feed pellet and b) a soluble composition comprising a therapeutically effective amount of monensin and a solvent is provided. The solvents, concentrations of monensin (i.e., weight of monensin/weight of monensin plus solvent), concentrations of solvent (i.e., weight of solvent/weight of solvent plus monensin), amounts of monensin, and adaptation for consumption provided in the preceding paragraphs are also applicable to the processes described herein.

FIG. 1 shows that food compositions including an alkaline component such as calcium hydroxide display a greater percentage of detectable monensin compared to food compositions that do not include calcium hydroxide.

The following embodiments are also contemplated:
1. A process for preparing an animal feed supplement comprising the step of combining a) a mineral mix and b) a soluble composition comprising a therapeutically effective amount of monensin and a solvent,
   wherein the mineral mix is substantially free of monocalcium phosphate.
2. The process of clause 1 wherein the soluble composition is allowed to dry following combination with the mineral mix.
3. The process of clause 1 or clause 2 wherein the animal feed supplement is subsequently combined with monocalcium phosphate.
4. The process of clause 3 wherein the monocalcium phosphate is a solid.
5. The process of any one of clauses 1 to 4 wherein the animal feed supplement is subsequently combined with an animal feed.
6. The process of any one of clauses 1 to 5 wherein the solvent is selected from the group consisting of benzyl alcohol, oleic acid, propylene glycol, vitamin E, ethanol, glyceryl mono- and di-caprylate, and mixtures thereof.
7. The process of any one of clauses 1 to 6 wherein the solvent is benzyl alcohol.
8. The process of any one of clauses 1 to 6 wherein the solvent is oleic acid.
9. The process of any one of clauses 1 to 6 wherein the solvent is propylene glycol.
10. The process of any one of clauses 1 to 6 wherein the solvent is vitamin E.
11. The process of any one of clauses 1 to 6 wherein the solvent is ethanol.
12. The process of any one of clauses 1 to 6 wherein the solvent is glyceryl mono- and di-caprylate.
13. The process of any one of clauses 1 to 12 wherein monensin is present at a concentration of about 5% to about 40% (weight/weight).
14. The process of any one of clauses 1 to 13 wherein monensin is present at a concentration of about 5% to about 35% (weight/weight).
15. The process of any one of clauses 1 to 14 wherein monensin is present at a concentration of about 5% to about 30% (weight/weight).
16. The process of any one of clauses 1 to 15 wherein monensin is present at a concentration of about 5% to about 25% (weight/weight).
17. The process of any one of clauses 1 to 16 wherein monensin is present at a concentration of about 5% to about 20% (weight/weight).
18. The process of any one of clauses 1 to 17 wherein monensin is present at a concentration of about 10% to about 20% (weight/weight).
19. The process of any one of clauses 1 to 16 wherein monensin is present at a concentration of approximately 25% (weight/weight).
20. The process of any one of clauses 1 to 18 wherein monensin is present at a concentration of approximately 20% (weight/weight).
21. The process of any one of clauses 1 to 18 wherein monensin is present at a concentration of approximately 15% (weight/weight).
22. The process of any one of clauses 1 to 18 wherein monensin is present at a concentration of approximately 10% (weight/weight).
23. The process of any one of clauses 1 to 17 wherein monensin is present at a concentration of approximately 5% (weight/weight).
24. The process of any one of clauses 1 to 23 wherein the solvent is present at a concentration of about 60% to about 95% (weight/weight).
25. The process of any one of clauses 1 to 24 wherein the solvent is present at a concentration of about 70% to about 95% (weight/weight).
26. The process of any one of clauses 1 to 25 wherein the solvent is present at a concentration of about 75% to about 95% (weight/weight).
27. The process of any one of clauses 1 to 18 or clauses 20 to 26 wherein the solvent is present at a concentration of about 80% to about 95% (weight/weight).
28. The process of any one of clauses 1 to 18 or clauses 20 to 26 wherein the solvent is present at a concentration of about 80% to about 90% (weight/weight).
29. The process of any one of clauses 1 to 18 or clauses 20 to 28 wherein the solvent is present at a concentration of approximately 80% (weight/weight).
30. The process of any one of clauses 1 to 18 or clauses 21 to 28 wherein the solvent is present at a concentration of approximately 85% (weight/weight).
31. The process of any one of clauses 1 to 18 or clauses 22 to 28 wherein the solvent is present at a concentration of approximately 90% (weight/weight).
32. The process of any one of clauses 1 to 17 or clauses 23 to 27 wherein the solvent is present at a concentration of approximately 95% (weight/weight).
33. An animal feed supplement made by the process of any one of clauses 1 to 32.
34. The animal feed supplement of clause 33 wherein the animal feed supplement is adapted for consumption by a ruminant.
35. The animal feed supplement of clause 34 wherein the ruminant is a bovine.
36. The animal feed supplement of clause 34 wherein the ruminant is a goat.
37. The animal feed supplement of clause 33 wherein the animal feed supplement is adapted for consumption by an avian.
38. A process for preparing an animal food composition comprising the step of combining a) an animal feed and b) a soluble composition comprising a therapeutically effective amount of monensin and a solvent,
   wherein the animal feed is substantially free of monocalcium phosphate.
39. The process of clause 38 wherein the soluble composition is allowed to dry following combination with the animal feed.
40. The process of clause 38 or clause 39 wherein the animal food composition is subsequently combined with monocalcium phosphate.
41. The process of clause 40 wherein the monocalcium phosphate is a solid.
42. The process of any one of clauses 38 to 41 wherein the solvent is selected from the group consisting of benzyl alcohol, oleic acid, propylene glycol, vitamin E, ethanol, glyceryl mono- and di-caprylate, and mixtures thereof.
43. The process of any one of clauses 38 to 42 wherein the solvent is benzyl alcohol.
44. The process of any one of clauses 38 to 42 wherein the solvent is oleic acid.
45. The process of any one of clauses 38 to 42 wherein the solvent is propylene glycol.
46. The process of any one of clauses 38 to 42 wherein the solvent is vitamin E.
47. The process of any one of clauses 38 to 42 wherein the solvent is ethanol.
48. The process of any one of clauses 38 to 42 wherein the solvent is glyceryl mono- and di-caprylate.
49. The process of any one of clauses 38 to 48 wherein monensin is present at a concentration of about 5% to about 40% (weight/weight).
50. The process of any one of clauses 38 to 49 wherein monensin is present at a concentration of about 5% to about 35% (weight/weight).
51. The process of any one of clauses 38 to 50 wherein monensin is present at a concentration of about 5% to about 30% (weight/weight).
52. The process of any one of clauses 38 to 51 wherein monensin is present at a concentration of about 5% to about 25% (weight/weight).
53. The process of any one of clauses 38 to 52 wherein monensin is present at a concentration of about 5% to about 20% (weight/weight).
54. The process of any one of clauses 38 to 53 wherein monensin is present at a concentration of about 10% to about 20% (weight/weight).
55. The process of any one of clauses 38 to 52 wherein monensin is present at a concentration of approximately 25% (weight/weight).
56. The process of any one of clauses 38 to 54 wherein monensin is present at a concentration of approximately 20% (weight/weight).
57. The process of any one of clauses 38 to 54 wherein monensin is present at a concentration of approximately 15% (weight/weight).
58. The process of any one of clauses 38 to 54 wherein monensin is present at a concentration of approximately 10% (weight/weight).
59. The process of any one of clauses 38 to 53 wherein monensin is present at a concentration of approximately 5% (weight/weight).
60. The process of any one of clauses 38 to 59 wherein the solvent is present at a concentration of about 60% to about 95% (weight/weight).
61. The process of any one of clauses 38 to 60 wherein the solvent is present at a concentration of about 70% to about 95% (weight/weight).
62. The process of any one of clauses 38 to 61 wherein the solvent is present at a concentration of about 75% to about 95% (weight/weight).
63. The process of any one of clauses 38 to 54 or clauses 56 to 62 wherein the solvent is present at a concentration of about 80% to about 95% (weight/weight).
64. The process of any one of clauses 38 to 54 or clauses 56 to 63 wherein the solvent is present at a concentration of about 80% to about 90% (weight/weight).
65. The process of any one of clauses 38 to 54 or clauses 56 to 64 wherein the solvent is present at a concentration of approximately 80% (weight/weight).
66. The process of any one of clauses 38 to 54 or clauses 57 to 64 wherein the solvent is present at a concentration of approximately 85% (weight/weight).
67. The process of any one of clauses 38 to 54 or clauses 58 to 64 wherein the solvent is present at a concentration of approximately 90% (weight/weight).
68. The process of any one of clauses 38 to 53 or clauses 59 to 63 wherein the solvent is present at a concentration of approximately 95% (weight/weight).
69. An animal food composition made by the process of any one of clauses 38 to 68.
70. The animal food composition of clause 69 wherein the animal food composition is adapted for consumption by a ruminant.
71. The animal food composition of clause 70 wherein the ruminant is a bovine.
72. The animal food composition of clause 70 wherein the ruminant is a goat.
73. The animal food composition of clause 69 wherein the animal food composition is adapted for consumption by an avian.
74. A process for preparing an animal food composition comprising the step of combining a) an animal feed pellet and b) a soluble composition comprising a therapeutically effective amount of monensin and a solvent.
75. The process of clause 74 wherein the soluble composition is allowed to dry following combination with the animal feed pellet.
76. The process of clause 74 or clause 75 wherein the solvent is selected from the group consisting of benzyl alcohol, oleic acid, propylene glycol, vitamin E, ethanol, glyceryl mono- and di-caprylate, and mixtures thereof.
77. The process of any one of clauses 74 to 76 wherein the solvent is benzyl alcohol.
78. The process of any one of clauses 74 to 76 wherein the solvent is oleic acid.
79. The process of any one of clauses 74 to 76 wherein the solvent is propylene glycol.
80. The process of any one of clauses 74 to 76 wherein the solvent is vitamin E.
81. The process of any one of clauses 74 to 76 wherein the solvent is ethanol.
82. The process of any one of clauses 74 to 76 wherein the solvent is glyceryl mono- and di-caprylate.
83. The process of any one of clauses 74 to 82 wherein monensin is present at a concentration of about 5% to about 40% (weight/weight).
84. The process of any one of clauses 74 to 83 wherein monensin is present at a concentration of about 5% to about 35% (weight/weight).
85. The process of any one of clauses 74 to 84 wherein monensin is present at a concentration of about 5% to about 30% (weight/weight).
86. The process of any one of clauses 74 to 85 wherein monensin is present at a concentration of about 5% to about 25% (weight/weight).
87. The process of any one of clauses 74 to 86 wherein monensin is present at a concentration of about 5% to about 20% (weight/weight).
88. The process of any one of clauses 74 to 87 wherein monensin is present at a concentration of about 10% to about 20% (weight/weight).
89. The process of any one of clauses 74 to 86 wherein monensin is present at a concentration of approximately 25% (weight/weight).
90. The process of any one of clauses 74 to 88 wherein monensin is present at a concentration of approximately 20% (weight/weight).
91. The process of any one of clauses 74 to 88 wherein monensin is present at a concentration of approximately 15% (weight/weight).
92. The process of any one of clauses 74 to 88 wherein monensin is present at a concentration of approximately 10% (weight/weight).
93. The process of any one of clauses 74 to 87 wherein monensin is present at a concentration of approximately 5% (weight/weight).
94. The process of any one of clauses 74 to 93 wherein the solvent is present at a concentration of about 60% to about 95% (weight/weight).
95. The process of any one of clauses 74 to 94 wherein the solvent is present at a concentration of about 70% to about 95% (weight/weight).
96. The process of any one of clauses 74 to 95 wherein the solvent is present at a concentration of about 75% to about 95% (weight/weight).
97. The process of any one of clauses 74 to 88 or clauses 90 to 96 wherein the solvent is present at a concentration of about 80% to about 95% (weight/weight).
98. The process of any one of clauses 74 to 88 or clauses 90 to 97 wherein the solvent is present at a concentration of about 80% to about 90% (weight/weight).
99. The process of any one of clauses 74 to 88 or clauses 90 to 98 wherein the solvent is present at a concentration of approximately 80% (weight/weight).
100. The process of any one of clauses 74 to 88 or clauses 91 to 98 wherein the solvent is present at a concentration of approximately 85% (weight/weight).
101. The process of any one of clauses 74 to 88 or clauses 92 to 98 wherein the solvent is present at a concentration of approximately 90% (weight/weight).
102. The process of any one of clauses 74 to 88 or clauses 93 to 97 wherein the solvent is present at a concentration of approximately 95% (weight/weight).
103. An animal food composition made by the process of any one of clauses 74 to 102.
104. The animal food composition of clause 103 wherein the animal food composition is adapted for consumption by a ruminant.
105. The animal food composition of clause 104 wherein the ruminant is a bovine.
106. The animal food composition of clause 104 wherein the ruminant is a goat.
107. The animal food composition of clause 103 wherein the animal food composition is adapted for consumption by an avian.

The further embodiments are also contemplated:
1. An animal feed supplement comprising a therapeutically effective amount of monensin, a solvent, and an alkaline component, wherein the supplement is a mixture.
2. The animal feed supplement of clause 1 wherein the solvent is selected from the group consisting of benzyl alcohol, oleic acid, propylene glycol, vitamin E, ethanol, glyceryl mono- and di-caprylate, and mixtures thereof.
3. The animal feed supplement of clause 1 or clause 2 wherein the solvent is benzyl alcohol.
4. The animal feed supplement of clause 1 or clause 2 wherein the solvent is oleic acid.
5. The animal feed supplement of clause 1 or clause 2 wherein the solvent is propylene glycol.
6. The animal feed supplement of clause 1 or clause 2 wherein the solvent is vitamin E.
7. The animal feed supplement of clause 1 or clause 2 wherein the solvent is ethanol.
8. The animal feed supplement of clause 1 or clause 2 wherein the solvent is glyceryl mono- and di-caprylate.
9. The animal feed supplement of any one of clauses 1 to 8 wherein the alkaline component is selected from the group consisting of calcium hydroxide, sodium hydroxide, potassium hydroxide, and mixtures thereof.
10. The animal feed supplement of any one of clauses 1 to 9 wherein the alkaline component is calcium hydroxide.
11. The animal feed supplement of any one of clauses 1 to 9 wherein the alkaline component is sodium hydroxide.
12. The animal feed supplement of any one of clauses 1 to 9 wherein the alkaline component is potassium hydroxide.
13. The animal feed supplement of any one of clauses 1 to 12 wherein monensin is present at a concentration of about 5% to about 40% (weight/weight) of said mixture.
14. The animal feed supplement of any one of clauses 1 to 13 wherein monensin is present at a concentration of about 5% to about 35% (weight/weight) of said mixture.
15. The animal feed supplement of any one of clauses 1 to 14 wherein monensin is present at a concentration of about 5% to about 30% (weight/weight) of said mixture.
16. The animal feed supplement of any one of clauses 1 to 15 wherein monensin is present at a concentration of about 5% to about 25% (weight/weight) of said mixture.
17. The animal feed supplement of any one of clauses 1 to 16 wherein monensin is present at a concentration of about 5% to about 20% (weight/weight) of said mixture.
18. The animal feed supplement of any one of clauses 1 to 17 wherein monensin is present at a concentration of about 10% to about 20% (weight/weight) of said mixture.
19. The animal feed supplement of any one of clauses 1 to 16 wherein monensin is present at a concentration of approximately 25% (weight/weight) of said mixture.
20. The animal feed supplement of any one of clauses 1 to 18 wherein monensin is present at a concentration of approximately 20% (weight/weight) of said mixture.
21. The animal feed supplement of any one of clauses 1 to 18 wherein monensin is present at a concentration of approximately 15% (weight/weight) of said mixture.
22. The animal feed supplement of any one of clauses 1 to 18 wherein monensin is present at a concentration of approximately 10% (weight/weight) of said mixture.
23. The animal feed supplement of any one of clauses 1 to 17 wherein monensin is present at a concentration of approximately 5% (weight/weight) of said mixture.
24. The animal feed supplement of any one of clauses 1 to 23 wherein the solvent is present at a concentration of about 50% to about 94.9% (weight/weight) of said mixture.
25. The animal feed supplement of any one of clauses 1 to 24 wherein the solvent is present at a concentration of about 55% to about 94.9% (weight/weight) of said mixture.
26. The animal feed supplement of any one of clauses 1 to 25 wherein the solvent is present at a concentration of about 60% to about 94.9% (weight/weight) of said mixture.
27. The animal feed supplement of any one of clauses 1 to 26 wherein the solvent is present at a concentration of about 65% to about 94.9% (weight/weight) of said mixture.
28. The animal feed supplement of any one of clauses 1 to 27 wherein the solvent is present at a concentration of about 70% to about 94.9% (weight/weight) of said mixture.
29. The animal feed supplement of any one of clauses 1 to 28 wherein the solvent is present at a concentration of about 70% to about 89.9% (weight/weight) of said mixture.
30. The animal feed supplement of any one of clauses 1 to 18 or clauses 20 to 29 wherein the solvent is present at a concentration of approximately 78% (weight/weight) of said mixture.
31. The animal feed supplement of any one of clauses 1 to 18 or clauses 21 to 29 wherein the solvent is present at a concentration of approximately 83% (weight/weight) of said mixture.
32. The animal feed supplement of any one of clauses 1 to 18 or clauses 22 to 29 wherein the solvent is present at a concentration of approximately 88% (weight/weight) of said mixture.
33. The animal feed supplement of any one of clauses 1 to 17 or clauses 23 to 28 wherein the solvent is present at a concentration of approximately 93% (weight/weight) of said mixture.
34. The animal feed supplement of any one of clauses 1 to 33 wherein the alkaline component is present at a concentration of about 0.1% to about 10% (weight/weight) of said mixture.
35. The animal feed supplement of any one of clauses 1 to 34 wherein the alkaline component is present at a concentration of about 1% to about 8% (weight/weight) of said mixture.
36. The animal feed supplement of any one of clauses 1 to 35 wherein the alkaline component is present at a concentration of about 2% to about 6% (weight/weight) of said mixture.
37. The animal feed supplement of any one of clauses 1 to 36 wherein the alkaline component is present at a concentration of about 3% to about 5% (weight/weight) of said mixture.
38. The animal feed supplement of any one of clauses 1 to 34 wherein the alkaline component is present at a concentration of about 0.1% to about 1% (weight/weight) of said mixture.
39. The animal feed supplement of any one of clauses 1 to 34 or clause 38 wherein the alkaline component is present at a concentration of about 0.1% (weight/weight) of said mixture.
40. The animal feed supplement of any one of clauses 1 to 35 or clause 38 wherein the alkaline component is present at a concentration of about 1% (weight/weight) of said mixture.
41. The animal feed supplement of any one of clauses 1 to 36 wherein the alkaline component is present at a concentration of about 2% (weight/weight) of said mixture.
42. The animal feed supplement of any one of clauses 1 to 37 wherein the alkaline component is present at a concentration of about 5% (weight/weight) of said mixture.
43. The animal feed supplement of any one of clauses 1 to 42 wherein the supplement is consumed by an animal at a rate of about 50 milligrams of monensin to about 500 milligrams of monensin per day.
44. The animal feed supplement of any one of clauses 1 to 43 wherein the supplement is consumed by an animal at a rate of about 100 milligrams of monensin to about 400 milligrams of monensin per day.
45. The animal feed supplement of any one of clauses 1 to 44 wherein the supplement is consumed by the animal at a rate of approximately 100 milligrams of monensin per day.
46. The animal feed supplement of any one of clauses 1 to 44 wherein the supplement is consumed by the animal at a rate of approximately 200 milligrams of monensin per day.
47. The animal feed supplement of any one of clauses 1 to 44 wherein the supplement is consumed by the animal at a rate of approximately 250 milligrams of monensin per day.
48. The animal feed supplement of any one of clauses 1 to 44 wherein the supplement is consumed by the animal at a rate of approximately 300 milligrams of monensin per day.
49. The animal feed supplement of any one of clauses 1 to 48 wherein the supplement is associated with an improvement in consumption by an animal.
50. A food composition comprising a therapeutically effective amount of monensin, a solvent, an alkaline component, and an animal feed.
51. The food composition of clause 50 further comprising a mineral mix.
52. The food composition of clause 50 or clause 51 wherein the solvent is present in a residual amount.
53. The food composition of any one of clauses 50 to 52 wherein the solvent is selected from the group consisting of benzyl alcohol, oleic acid, propylene glycol, vitamin E, ethanol, glyceryl mono- and di-caprylate, and mixtures thereof.
54. The food composition of any one of clauses 50 to 53 wherein the solvent is benzyl alcohol.
55. The food composition of any one of clauses 50 to 53 wherein the solvent is oleic acid.
56. The food composition of any one of clauses 50 to 53 wherein the solvent is propylene glycol.
57. The food composition of any one of clauses 50 to 53 wherein the solvent is vitamin E.
58. The food composition of any one of clauses 50 to 53 wherein the solvent is ethanol.
59. The food composition of any one of clauses 50 to 53 wherein the solvent is glyceryl mono- and di-caprylate.
60. The food composition of any one of clauses 50 to 59 wherein the alkaline component is selected from the group consisting of calcium hydroxide, sodium hydroxide, potassium hydroxide, and mixtures thereof.
61. The food composition of any one of clauses 50 to 60 wherein the alkaline component is calcium hydroxide.
62. The food composition of any one of clauses 50 to 60 wherein the alkaline component is sodium hydroxide.
63. The food composition of any one of clauses 50 to 60 wherein the alkaline component is potassium hydroxide.
64. The food composition of any one of clauses 50 to 63 wherein monensin is present at a concentration of about 5% to about 40% (weight/weight).
65. The food composition of any one of clauses 50 to 64 wherein monensin is present at a concentration of about 5% to about 35% (weight/weight).
66. The food composition of any one of clauses 50 to 65 wherein monensin is present at a concentration of about 5% to about 30% (weight/weight).
67. The food composition of any one of clauses 50 to 66 wherein monensin is present at a concentration of about 5% to about 25% (weight/weight).
68. The food composition of any one of clauses 50 to 67 wherein monensin is present at a concentration of about 5% to about 20% (weight/weight).
69. The food composition of any one of clauses 50 to 68 wherein monensin is present at a concentration of about 10% to about 20% (weight/weight).
70. The food composition of any one of clauses 50 to 67 wherein monensin is present at a concentration of approximately 25% (weight/weight).
71. The food composition of any one of clauses 50 to 69 wherein monensin is present at a concentration of approximately 20% (weight/weight).
72. The food composition of any one of clauses 50 to 69 wherein monensin is present at a concentration of approximately 15% (weight/weight).
73. The food composition of any one of clauses 50 to 69 wherein monensin is present at a concentration of approximately 10% (weight/weight).
74. The food composition of any one of clauses 50 to 68 wherein monensin is present at a concentration of approximately 5% (weight/weight).
75. The food composition of any one of clauses 50 to 74 wherein the solvent is present at a concentration of about 50% to about 94.9% (weight/weight).
76. The food composition of any one of clauses 50 to 75 wherein the solvent is present at a concentration of about 55% to about 94.9% (weight/weight).
77. The food composition of any one of clauses 50 to 76 wherein the solvent is present at a concentration of about 60% to about 94.9% (weight/weight).
78. The food composition of any one of clauses 50 to 77 wherein the solvent is present at a concentration of about 65% to about 94.9% (weight/weight).
79. The food composition of any one of clauses 50 to 78 wherein the solvent is present at a concentration of about 70% to about 94.9% (weight/weight).
80. The food composition of any one of clauses 50 to 79 wherein the solvent is present at a concentration of about 70% to about 89.9% (weight/weight).
81. The food composition of any one of clauses 50 to 69 or clauses 71 to 80 wherein the solvent is present at a concentration of approximately 78% (weight/weight).
82. The food composition of any one of clauses 50 to 69 or clauses 72 to 80 wherein the solvent is present at a concentration of approximately 83% (weight/weight).
83. The food composition of any one of clauses 50 to 69 or clauses 73 to 80 wherein the solvent is present at a concentration of approximately 88% (weight/weight).
84. The food composition of any one of clauses 50 to 68 or clauses 74 to 79 wherein the solvent is present at a concentration of approximately 93% (weight/weight).
85. The food composition of any one of clauses 50 to 84 wherein the alkaline component is present at a concentration of about 0.1% to about 10% (weight/weight).
86. The food composition of any one of clauses 50 to 85 wherein the alkaline component is present at a concentration of about 1% to about 8% (weight/weight).
87. The food composition of any one of clauses 50 to 86 wherein the alkaline component is present at a concentration of about 2% to about 6% (weight/weight).
88. The food composition of any one of clauses 50 to 87 wherein the alkaline component is present at a concentration of about 3% to about 5% (weight/weight).
89. The food composition of any one of clauses 50 to 85 wherein the alkaline component is present at a concentration of about 0.1% to about 1% (weight/weight).
90. The food composition of any one of clauses 50 to 85 or clause 89 wherein the alkaline component is present at a concentration of about 0.1% (weight/weight).
91. The food composition of any one of clauses 50 to 86 or clause 89 wherein the alkaline component is present at a concentration of about 1% (weight/weight).
92. The food composition of any one of clauses 50 to 87 wherein the alkaline component is present at a concentration of about 2% (weight/weight).
93. The food composition of any one of clauses 50 to 88 wherein the alkaline component is present at a concentration of about 5% (weight/weight).
94. The food composition of any one of clauses 50 to 93 wherein the composition is consumed by an animal at a rate of about 50 milligrams of monensin to about 500 milligrams of monensin per day.
95. The food composition of any one of clauses 50 to 94 wherein the composition is consumed by an animal at a rate of about 100 milligrams of monensin to about 400 milligrams of monensin per day.
96. The food composition of any one of clauses 50 to 95 wherein the composition is consumed by an animal at a rate of approximately 100 milligrams of monensin per day.
97. The food composition of any one of clauses 50 to 95 wherein the composition is consumed by an animal at a rate of approximately 200 milligrams of monensin per day.
98. The food composition of any one of clauses 50 to 95 wherein the composition is consumed by an animal at a rate of approximately 250 milligrams of monensin per day.
99. The food composition of any one of clauses 50 to 95 wherein the composition is consumed by an animal at a rate of approximately 300 milligrams of monensin per day.
100. The food composition of any one of clauses 50 to 99 wherein the composition is associated with an improvement in consumption by an animal.
101. A method of improving feed efficiency comprising administering to an animal in need thereof a food composition comprising a therapeutically effective amount of monensin, a solvent, an alkaline component, and an animal feed.
102. A method of increasing milk production efficiency comprising administering to an animal in need the thereof a food composition comprising a therapeutically effective amount of monensin, a solvent, an alkaline component, and an animal feed.
103. A method of increasing rate of weight gain comprising administering to an animal in need thereof a food composition comprising a therapeutically effective amount of monensin, a solvent, an alkaline component, and an animal feed.
104. A method of preventing or treating of coccidiosis comprising administering to an animal in need thereof a food composition comprising a therapeutically effective amount of monensin, a solvent, an alkaline component, and an animal feed.
105. The method of clause 104 wherein the coccidiosis is caused by a species of Eimeria.
106. The method of clause 104 or clause 105 wherein the coccidiosis is caused by Eimeria bovis.
107. The method of clause 104 or clause 105 wherein the coccidiosis is caused by Eimeria zuernii.
108. The method of clause 104 or clause 105 wherein the coccidiosis is caused by Eimeria crandallis.
109. The method of clause 104 or clause 105 wherein the coccidiosis is caused by Eimeria christenseni.
110. The method of clause 104 or clause 105 wherein the coccidiosis is caused by Eimeria ninakohlyakimovae.
111. The method of any one of clauses 101 to 110 further comprising a mineral mix.
112. The method of any one of clauses 101 to 111 wherein the solvent is present in a residual amount.
113. The method of any one of clauses 101 to 112 wherein the animal is a ruminant.
114. The method of clause 113 wherein the ruminant is a bovine.
115. The method of clause 113 wherein the ruminant is a goat.
116. The method of any one of clauses 101 to 112 wherein the animal is an avian.
117. The method of any one of clauses 101 to 116 wherein the solvent is selected from the group consisting of benzyl alcohol, oleic acid, propylene glycol, vitamin E, ethanol, glyceryl mono- and di-caprylate, and mixtures thereof.
118. The method of any one of clauses 101 to 117 wherein the solvent is benzyl alcohol.
119. The method of any one of clauses 101 to 117 wherein the solvent is oleic acid.
120. The method of any one of clauses 101 to 117 wherein the solvent is propylene glycol.
121. The method of any one of clauses 101 to 117 wherein the solvent is vitamin E.
122. The method of any one of clauses 101 to 117 wherein the solvent is ethanol.
123. The method of any one of clauses 101 to 117 wherein the solvent is glyceryl mono- and di-caprylate.
124. The method of any one of clauses 101 to 123 wherein the alkaline component is selected from the group consisting of calcium hydroxide, sodium hydroxide, potassium hydroxide, and mixtures thereof.
125. The method of any one of clauses 101 to 124 wherein the alkaline component is calcium hydroxide.
126. The method of any one of clauses 101 to 124 wherein the alkaline component is sodium hydroxide.
127. The method of any one of clauses 101 to 124 wherein the alkaline component is potassium hydroxide.
128. The method of any one of clauses 101 to 127 wherein monensin is present at a concentration of about 5% to about 40% (weight/weight).
129. The method of any one of clauses 101 to 128 wherein monensin is present at a concentration of about 5% to about 35% (weight/weight).
130. The method of any one of clauses 101 to 129 wherein monensin is present at a concentration of about 5% to about 30% (weight/weight).
131. The method of any one of clauses 101 to 130 wherein monensin is present at a concentration of about 5% to about 25% (weight/weight).
132. The method of any one of clauses 101 to 131 wherein monensin is present at a concentration of about 5% to about 20% (weight/weight).
133. The method of any one of clauses 101 to 132 wherein monensin is present at a concentration of about 10% to about 20% (weight/weight).
134. The method of any one of clauses 101 to 131 wherein monensin is present at a concentration of approximately 25% (weight/weight).
135. The method of any one of clauses 101 to 133 wherein monensin is present at a concentration of approximately 20% (weight/weight).
136. The method of any one of clauses 101 to 133 wherein monensin is present at a concentration of approximately 15% (weight/weight).
137. The method of any one of clauses 101 to 133 wherein monensin is present at a concentration of approximately 10% (weight/weight).
138. The method of any one of clauses 101 to 132 wherein monensin is present at a concentration of approximately 5% (weight/weight).
139. The method of any one of clauses 101 to 138 wherein the solvent is present at a concentration of about 50% to about 94.9% (weight/weight).
140. The method of any one of clauses 101 to 139 wherein the solvent is present at a concentration of about 55% to about 94.9% (weight/weight).
141. The method of any one of clauses 101 to 140 wherein the solvent is present at a concentration of about 60% to about 94.9% (weight/weight).
142. The method of any one of clauses 101 to 141 wherein the solvent is present at a concentration of about 65% to about 94.9% (weight/weight).
143. The method of any one of clauses 101 to 142 wherein the solvent is present at a concentration of about 70% to about 94.9% (weight/weight).
144. The method of any one of clauses 101 to 143 wherein the solvent is present at a concentration of about 70% to about 89.9% (weight/weight).
145. The method of any one of clauses 101 to 133 or clauses 135 to 144 wherein the solvent is present at a concentration of approximately 78% (weight/weight).
146. The method of any one of clauses 101 to 133 or clauses 136 to 144 wherein the solvent is present at a concentration of approximately 83% (weight/weight).
147. The method of any one of clauses 101 to 133 or clauses 137 to 144 wherein the solvent is present at a concentration of approximately 88% (weight/weight).
148. The method of any one of clauses 101 to 132 or clauses 138 to 143 wherein the solvent is present at a concentration of approximately 93% (weight/weight).
149. The method of any one of clauses 101 to 148 wherein the alkaline component is present at a concentration of about 0.1% to about 10% (weight/weight).
150. The method of any one of clauses 101 to 149 wherein the alkaline component is present at a concentration of about 1% to about 8% (weight/weight).
151. The method of any one of clauses 101 to 150 wherein the alkaline component is present at a concentration of about 2% to about 6% (weight/weight).
152. The method of any one of clauses 101 to 151 wherein the alkaline component is present at a concentration of about 3% to about 5% (weight/weight).
153. The method of any one of clauses 101 to 150 wherein the alkaline component is present at a concentration of about 0.1% to about 1% (weight/weight).
154. The method of any one of clauses 101 to 149 or clause 153 wherein the alkaline component is present at a concentration of about 0.1% (weight/weight).
155. The method of any one of clauses 101 to 150 or clause 153 wherein the alkaline component is present at a concentration of about 1% (weight/weight).
156. The method of any one of clauses 101 to 151 wherein the alkaline component is present at a concentration of about 2% (weight/weight).
157. The method of any one of clauses 101 to 152 wherein the alkaline component is present at a concentration of about 5% (weight/weight).
158. The method of any one of clauses 101 to 157 wherein the composition is consumed by the animal at a rate of about 50 milligrams of monensin to about 500 milligrams of monensin per day.
159. The method of any one of clauses 101 to 158 wherein the composition is consumed by the animal at a rate of about 100 milligrams of monensin to about 400 milligrams of monensin per day.
160. The method of any one of clauses 101 to 159 wherein the composition is consumed by the animal at a rate of approximately 100 milligrams of monensin per day.
161. The method of any one of clauses 101 to 159 wherein the composition is consumed by the animal at a rate of approximately 200 milligrams of monensin per day.
162. The method of any one of clauses 101 to 159 wherein the composition is consumed by the animal at a rate of approximately 250 milligrams of monensin per day.
163. The method of any one of clauses 101 to 159 wherein the composition is consumed by the animal at a rate of approximately 300 milligrams of monensin per day.
164. The method of any one of clauses 101 to 163 wherein the method is associated with an improvement in consumption of the composition by the animal.
165. A process for preparing a food composition comprising combining a) a mixture comprising monensin and a solvent, b) an alkaline component, and c) an animal feed.
166. The process of clause 165 further comprising combining with d) a mineral mix.
167. The process of clause 165 or clause 166 wherein the solvent is selected from the group consisting of benzyl alcohol, oleic acid, propylene glycol, vitamin E, ethanol, glyceryl mono- and di-caprylate, and mixtures thereof.
168. The process of any one of clauses 165 to 167 wherein the solvent is benzyl alcohol.
169. The process of any one of clauses 165 to 167 wherein the solvent is oleic acid.
170. The process of any one of clauses 165 to 167 wherein the solvent is propylene glycol.
171. The process of any one of clauses 165 to 167 wherein the solvent is vitamin E.
172. The process of any one of clauses 165 to 167 wherein the solvent is ethanol.
173. The process of any one of clauses 165 to 167 wherein the solvent is glyceryl mono- and di-caprylate.
174. The process of any one of clauses 165 to 173 wherein the alkaline component is selected from the group consisting of calcium hydroxide, sodium hydroxide, potassium hydroxide, and mixtures thereof.
175. The process of any one of clauses 165 to 174 wherein the alkaline component is calcium hydroxide.
176. The process of any one of clauses 165 to 174 wherein the alkaline component is sodium hydroxide.
177. The process of any one of clauses 165 to 174 wherein the alkaline component is potassium hydroxide.
178. The process of any one of clauses 165 to 177 wherein monensin is present at a concentration of about 5% to about 40% (weight/weight).
179. The process of any one of clauses 165 to 178 wherein monensin is present at a concentration of about 5% to about 35% (weight/weight).
180. The process of any one of clauses 165 to 179 wherein monensin is present at a concentration of about 5% to about 30% (weight/weight).
181. The process of any one of clauses 165 to 180 wherein monensin is present at a concentration of about 5% to about 25% (weight/weight).
182. The process of any one of clauses 165 to 181 wherein monensin is present at a concentration of about 5% to about 20% (weight/weight).
183. The process of any one of clauses 165 to 182 wherein monensin is present at a concentration of about 10% to about 20% (weight/weight).
184. The process of any one of clauses 165 to 181 wherein monensin is present at a concentration of approximately 25% (weight/weight).
185. The process of any one of clauses 165 to 183 wherein monensin is present at a concentration of approximately 20% (weight/weight).
186. The process of any one of clauses 165 to 183 wherein monensin is present at a concentration of approximately 15% (weight/weight).
187. The process of any one of clauses 165 to 183 wherein monensin is present at a concentration of approximately 10% (weight/weight).
188. The process of any one of clauses 165 to 182 wherein monensin is present at a concentration of approximately 5% (weight/weight).
189. The process of any one of clauses 165 to 188 wherein the solvent is present at a concentration of about 50% to about 94.9% (weight/weight).
190. The process of any one of clauses 165 to 189 wherein the solvent is present at a concentration of about 55% to about 94.9% (weight/weight).
191. The process of any one of clauses 165 to 190 wherein the solvent is present at a concentration of about 60% to about 94.9% (weight/weight).
192. The process of any one of clauses 165 to 191 wherein the solvent is present at a concentration of about 65% to about 94.9% (weight/weight).
193. The process of any one of clauses 165 to 192 wherein the solvent is present at a concentration of about 70% to about 94.9% (weight/weight).
194. The process of any one of clauses 165 to 193 wherein the solvent is present at a concentration of about 70% to about 89.9% (weight/weight).
195. The process of any one of clauses 165 to 183 or clauses 185 to 194 wherein the solvent is present at a concentration of approximately 78% (weight/weight).
196. The process of any one of clauses 165 to 183 or clauses 186 to 194 wherein the solvent is present at a concentration of approximately 83% (weight/weight).
197. The process of any one of clauses 165 to 183 or clauses 187 to 194 wherein the solvent is present at a concentration of approximately 88% (weight/weight).
198. The process of any one of clauses 165 to 182 or clauses 188 to 193 wherein the solvent is present at a concentration of approximately 93% (weight/weight).
199. The process of any one of clauses 165 to 198 wherein the alkaline component is present at a concentration of about 0.1% to about 10% (weight/weight).
200. The process of any one of clauses 165 to 199 wherein the alkaline component is present at a concentration of about 1% to about 8% (weight/weight).
201. The process of any one of clauses 165 to 200 wherein the alkaline component is present at a concentration of about 2% to about 6% (weight/weight).
202. The process of any one of clauses 165 to 201 wherein the alkaline component is present at a concentration of about 3% to about 5% (weight/weight).
203. The process of any one of clauses 165 to 199 wherein the alkaline component is present at a concentration of about 0.1% to about 1% (weight/weight).
204. The process of any one of clauses 165 to 199 or clause 203 wherein the alkaline component is present at a concentration of about 0.1% (weight/weight).
205. The process of any one of clauses 165 to 200 or clause 203 wherein the alkaline component is present at a concentration of about 1% (weight/weight).
206. The process of any one of clauses 165 to 201 wherein the alkaline component is present at a concentration of about 2% (weight/weight).
207. The process of any one of clauses 165 to 202 wherein the alkaline component is present at a concentration of about 5% (weight/weight).

### EXAMPLE 1

### Consumption of A Monensin-Containing Food Composition

Consumption of the food composition of the present disclosure can be evaluated in bovines. Three groups of bovines can be evaluated in the present example. In one group, the food composition of the present disclosure containing monensin, a solvent, and an animal feed can be combined with a mineral mix fed to bovines. In a second group, a solid formulation of monensin can be combined with a mineral mix and an animal feed and fed to bovines. In a third group, a mineral mix and an animal feed can be combined as a control formulation and fed to bovines. The average intake of the combination (grams per day) for each group can be evaluated, as well as the average daily weight gain (kilograms per day). Table 1 shows the results of the study. Bovines fed the food composition of the present disclosure containing monensin, a solvent, and an animal feed can display a numerically superior intake of the combination and a greater intake compared to bovines fed the solid formulation of monensin combined with a mineral mix and an animal feed.

TABLE 1

| | | | Weekly Mineral |
|---|---|---|---|
| | | | Intake (g of |
| | Average Daily | Mineral Intake | mineral/kg of |
| Group | Gain (kg/day) | (g/day) | bodyweight) |
| Control | −0.04 | 71.5 | 1.18 |
| Food Composition | −0.16 | 56.0 (−22%)* | 0.92* |
| Prepared with | | | |
| Solid Formulation | | | |
| of Monensin | | | |
| Food Composition | −0.01 | 64.7 (−10%) | 1.07 |
| Prepared with | | | |
| Monensin | | | |
| Formulation of | | | |
| Present Disclosure | | | |

* = p < 0.05 vs. control

### EXAMPLE 2

### Detection of Monensin In Food Compositions With And Without Alkaline Material

The detection of monensin in food compositions of the present disclosure can be evaluated. In one group, a food composition comprising monensin, a solvent, an alkaline component, and an animal feed can be assayed for the detection of monensin. In the first group, the solvent can be benzyl alcohol and the alkaline component can be calcium hydroxide. In another group, a food composition comprising monensin, a solvent, and an animal feed can be assayed for the detection of monensin. FIG. 1 shows the results of this study. Over time, the food composition including calcium hydroxide can display a greater percentage of detectable monensin compared to the food composition that does not include calcium hydroxide.

1-34. (canceled)

35. An animal feed supplement for use in an animal feed composition having monocalcium phosphate therein, comprising monensin, one or more solvents, and an alkaline component, wherein the supplement is a mixture, and wherein said alkaline component is present in said animal feed supplement in an amount sufficient to reducing the formation of a monocalcium phosphate-monensin complex in a liquid environment.

36. The animal feed supplement of claim 35, wherein the solvent is-selected from benzyl alcohol, oleic acid, propylene glycol, vitamin E, ethanol, glyceryl mono- and di-caprylate, and mixtures thereof, and the alkaline component is calcium hydroxide, sodium hydroxide, potassium hydroxide, and mixtures thereof.

37. The animal feed supplement of claim 35, wherein the alkaline component is present in the supplement in an amount of between about 0.1% to about 10% (weight of alkaline component/(weight of alkaline component plus monensin plus solvent)); and the solvent is present in the supplement in an amount of between about 50% to about 94.9% (weight of solvent/(weight of solvent plus monensin plus alkaline component)).

38. An animal feed composition having monocalcium phosphate therein, comprising an animal feed supplement of claim 35 and an animal feed.

39. The animal feed composition of claim 38, wherein the solvent is allowed to evaporate such that said solvent is present in a residual amount.

40. The animal feed composition of claim 38, wherein said animal feed is a mineral mix having monocalcium phosphate therein.

41. A method of reducing the formation of a monocalcium phosphate-monensin complex in a liquid environment when monensin, in one or more solvents, is added to an animal feed composition having monocalcium phosphate therein, the method comprising including an alkaline component in said liquid environment sufficient to reduce the formation of said complex.

42. The method of claim 41, wherein the alkaline component is part of animal feed supplement comprising monensin, one or more solvents, and an alkaline component, wherein the supplement is a mixture.
